(19) United States
(12) Patent Application Publication
(10) Pub. No.: US 2024/0247291 A1
(43) Pub. Date: Jul. 25, 2024

(54) COMB POLYELECTROLYTE STABILIZED COMPLEX COACERVATE EMULSIONS

(71) Applicant: The Regents of the University of California, Oakland, CA (US)

(72) Inventors: Samanvaya Srivastava, Los Angeles, CA (US); Shang Gao, Fremont, CA (US); Advait Suhas Holkar, Los Angeles, CA (US)

(73) Assignee: The Regents of the University of California, Oakland, CA (US)

(21) Appl. No.: 18/559,983

(22) PCT Filed: May 11, 2022
(86) PCT No.: PCT/US22/28766

§ 371 Date: Nov. 9, 2023

Related U.S. Application Data
(60) Provisional application No. 63/187,031, filed on May 11, 2021.

(51) Int. Cl. C12P 19/00; A01N 25/04; A61K 47/30; B01J 13/10; C12N 9/02; C12N 9/20

(52) U.S. Cl. CPC C12P 19/00; A01N 25/04; A61K 47/30; B01J 13/10; C12N 9/0008; C12N 9/20; C12Y 102/07; C12Y 301/01003

(57) ABSTRACT

The invention described herein provides methods and materials that can impart long-term stability to polyelectrolyte complex coacervate droplets and create complex coacervate emulsions. The methodology described herein is designed to use one or more of a wide variety of comb polyelectrolytes in order to produce stable water-in-water emulsions with precisely controlled droplet size and enhanced stability profiles. The stabilized water-in-water emulsions microdroplets of the invention can further encapsulate active agents such as proteins and the like in a manner that protects them from the surrounding environment, thus allowing the compositions to serve as bio-microreactors and the like.

### CROSS-REFERENCE TO RELATED APPLICATIONS

This application claims the benefit under 35 U.S.C. Section 119(e) of co-pending and commonly-assigned U.S. Provisional Patent Application Ser. No. 63/187,031, filed on May 11, 2021, and entitled “COMB POLYELECTROLYTE STABILIZED COMPLEX COACERVATE EMULSIONS” which application is incorporated by reference herein.

### TECHNICAL FIELD

The present invention relates to complex coacervate compositions and methods for making and using them.

### BACKGROUND OF THE INVENTION

Complex coacervation is a liquid-liquid phase separation phenomenon that occurs through electrostatic complexation of oppositely charged macromolecules (e.g., synthetic polyelectrolytes, and biopolyelectrolytes including DNA and RNA, as well as charged biomacromolecules such as proteins, macroions and the like) and subsequent condensation into a macromolecule-rich phase. Upon mixing of oppositely charged macromolecules in an aqueous solution, complex coacervate microdroplets appear spontaneously. These droplets possess a distinct water-water interface with the ambient aqueous environments and have a strong propensity to partition and encapsulate charge-bearing molecules (e.g., proteins, multivalent ions, nucleic acids and the like). As such, they have been presented as minimalistic membraneless protocells exhibiting dynamic spatial compartmentalization that are capable of efficiently and spontaneously sequestering biological molecules and enhancing their activity. However, the lack of a membrane around the coacervate droplets also makes them prone to coalescence and Ostwald ripening, leading to macrophase separation in coacervate solutions. The lack of long-term droplet stability significantly hampers the use of coacervates as protocells, bioreactors, or encapsulants.

For the reasons noted above, there is a need in the art for stabilized coacervate compositions and methods for making and using them.

### SUMMARY OF THE INVENTION

The invention described herein provides methods and materials that can impart long-term stability to polyelectrolyte complex coacervate droplets and create complex coacervate emulsions. Embodiments of the methods described herein are designed to use comb polyelectrolyte compounds to produce stable water-in-water emulsions with precisely controlled droplet size and enhanced stability profiles. The stabilized water-in-water emulsions microdroplets of the invention can further encapsulate active agents such as proteins and the like in a manner that protects them from the surrounding environment, thus allowing the compositions to serve as bio-microreactors in certain embodiments of the invention. These water-in-water emulsions droplets can also be utilized in a wide variety of other ways, for example to produce compositions for hair conditioner formulations that provide improved wet and dry hair compatibility. In addition, embodiments of the stable complex coacervate emulsions disclosed herein are also ideal for uses in environmental protection, for example as agents for stabilizing pesticides, as well as anti-erosion agents.

As discussed below, we have developed methods and materials for stabilizing coacervate microdroplets via the use of a constellation of ingredients which include comb polyelectrolytes in complex coacervate formulations. This methodology produces stable complex coacervate emulsions comprising microdroplets, composed of oppositely charged linear polyelectrolytes and stabilized by interfacially adsorbed comb polyelectrolytes, that exhibit long-term (>4+months) stability. The microdroplet size of these complex coacervate emulsions remains relatively constant with time and is shown to be regulated by the concentrations of the comb and linear polyelectrolytes. Embodiments of this stabilization strategy improve the salt resistance of the complex coacervates while minimally interfering with other desirable attributes of coacervate droplets, including their ability to sequester and encapsulate proteins from solution, as well as their membraneless interface, an interface that allows for fast transport of small molecules and the like across them.

The invention disclosed herein has a number of embodiments. Embodiments of the invention include, for example, compositions of matter including water; a water-soluble comb polyelectrolyte; a positively charged water-soluble macromolecule; and a negatively charged water-soluble macromolecule; wherein the water-soluble comb polyelectrolyte, the positively charged water-soluble macromolecule and the negatively charged water-soluble macromolecule in the composition form complex coacervate droplets. In typical embodiments of the invention, the water-soluble comb polyelectrolyte comprises an anionic comb polyelectrolyte; the positively charged water-soluble macromolecule comprises a linear polyelectrolyte; and the negatively charged water-soluble macromolecule comprises a linear polyelectrolyte. In certain embodiments of the invention, the composition further comprises at least one additional agent such as a polypeptide; a polynucleotide; a protein; a therapeutic agent; a diagnostic agent; a pesticide; a macroion; a pharmaceutical excipient; and/or a salt.

Embodiments of the invention also include methods of making the complex coacervate droplets disclosed herein. Typically, these methods comprise combining together water; a water-soluble comb polyelectrolyte; a positively charged water-soluble macromolecule; and a negatively charged water-soluble macromolecule. In such methods the water-soluble comb polyelectrolyte, the positively charged water-soluble macromolecule and the negatively charged water-soluble macromolecule are selected to have material properties and used in selected amounts/ratios in the composition so that they combine together in the water to form a complex coacervate. Some embodiments of these methods include further disposing in the complex coacervate at least one of: a polypeptide; a polynucleotide; a protein; a therapeutic agent; a diagnostic agent; a macroion; a pesticide; a pharmaceutical excipient; a salt or the like.

In illustrative methods of making a complex coacervate, the comb polyelectrolyte is selected to have material properties that allow it to adsorb on complex coacervate droplet surfaces (water-water interfaces) so as to provide steric stabilization to the droplets. In certain of the methods of making a complex coacervate, the method forms microdroplets having a mean diameter from 0.05 μm to 10 μm; and/or the complex coacervate formed in the method exhibits turbidity at concentrations of 500 mM NaCl, and/or the complex coacervate formed in the method remains stable for 48 hours following a >120-fold dilution from a concentrated single-phase solution of the complex coacervate. In some embodiments of the invention, the complex coacervate formed in the method spontaneously encapsulates polypeptides disposed in the composition. Embodiments of the invention further include complex coacervates made by the methods disclosed herein.

Other embodiments of the invention include methods of using the compositions disclosed herein to stabilize an activity of a molecule and/or inhibit the degradation of the molecule. Such methods comprise disposing the molecule as a cargo in a microdroplet of a complex coacervate composition disclosed herein, such that an activity of the cargo molecule is stabilized and/or its degradation is inhibited. Embodiments of the invention also include methods of performing a biochemical reaction, the methods comprising disposing molecules that perform the biological reaction (e.g. one or more enzymes such as an oxidoreductase; a transferase; a hydrolase; a lyase; a isomerase or a ligase) as a cargo within a complex coacervate formed to comprise water; a water-soluble comb polyelectrolyte; a positively charged water-soluble macromolecule; and a negatively charged water-soluble macromolecule; such that the biochemical reaction is performed within the complex coacervate. In certain embodiments of the invention, the cargo comprises a plurality of enzymes; and the product generated by a first enzyme in the plurality of enzymes is a reactant for a second enzyme within the plurality of enzymes (e.g., so as to perform an enzyme cascade within the complex coacervate composition).

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1: Comb polyelectrolyte dispersants stabilize otherwise unstable polyelectrolyte complex coacervate droplets that are prone to coalescence. (a) Columns 1 and 2: Complex coacervate droplets obtained upon mixing of solutions oppositely charged linear polyelectrolytes (total polyelectrolyte concentrations 15.9-62.2 mM, polyelectrolyte charge matched solutions). Turbidity in the as-mixed solutions (column 1) emerges from the presence of complex coacervate microdroplets. These complex coacervate microdroplets are prone to coalescence and merge into a homogenous transparent coacervate phase after ˜3 hours, with the polymer dense phase coacervate phase at the bottom and the polymer-lean supernatant phase at the top (column 2). Columns 3-7: Stable complex coacervate emulsions obtained by preparing the same solutions as in Columns 1 and 2 with an additional component: 4.8 mM anionic comb polyelectrolytes (MasterGlenium 7500). These stable complex coacervate emulsions are turbid when prepared (column 3) and maintain a uniformly turbidity for more than 48 hours (column 4). Over 4 months of storage, the complex coacervate microdroplets settle to the bottom of the vial but do not coalesce, leading to a dense emulsion at the bottom of the vial (column 5). Upon gentle shaking of the vial, the microdroplets re-disperse, resulting in turbid emulsions (column 6). The shaken vials remain turbid for the next 48 hours (column 7), indicating no major changes in the stability of the emulsions. Turbidity in the samples is a key factor for indicating the stability of the emulsions. (b) Micrographs of the as-prepared unstable complex coacervates droplets (column 1) and stabilized complex coacervate emulsions (column 2). The stabilized emulsions contain 4.8 mM comb polyclectrolyte dispersants in addition to the oppositely charged linear polyelectrolytes. Both as-mixed solutions contain micron-sized droplets. The unstable emulsions undergo droplet coalescence, while the stabilized coacervate emulsions maintain droplet stability, as evident from micrographs of the stabilized emulsions visualized after 48 hours after mixing (column 3). Scale bars are provided in each micrograph. The linear polyelectrolytes are polydiallydimethylammonium chloride (pDADMAC) (molecular weight 8500 g/mol) and polyacrylic acid (PAA) (molecular weight 5100 g/mol).

FIG. 2: Microdroplet size in stable complex coacervate emulsions is controlled by the linear polyelectrolyte and the comb polyelectrolyte concentrations. (a) Stable polyelectrolyte complex coacervate emulsions are prepared at 155.3 mM charge concentration of between oppositely charged linear polyelectrolytes (2 wt % as total polyelectrolyte concentration by weight, 1:1 charge ratio) with increasing concentration, from 0 mM to 19.1 mM charge, of anionic comb polyelectrolyte. Photographs are taken from different times, indicated as “as mixed”, 3 hours, 24 hours, and 48 hours after mixing. Turbidity in the samples is a key factor for indicating the stability of the emulsions. (b) The evolution of the mean diameter (by volume) of the complex coacervate microdroplets with time in formulations comprising total polyelectrolyte concentrations ranging from 15.9 mM to 155.3 mM (polyelectrolyte charge) and increasing anionic comb polyelectrolyte concentration. Microdroplet size increased with increasing linear polyelectrolyte concentrations but undergoes a nonmonotonic trend with comb polyelectrolyte concentration. The microdroplet size were measured using dynamic light scattering and averaged from triplicate samples at each concentration and three measurement trials for each sample. Filled symbols denote data measured from unperturbed samples. Open symbols denote data measured from samples that were shaken gently. Errors are calculated through the mean standard deviations of the measurements. Error bars are shown when they are larger than the symbols. The linear polyelectrolytes are polydiallydimethylammonium chloride (pDADMAC) (molecular weight 8500 g/mol) and polyacrylic acid (PAA) (molecular weight 5100 g/mol). The comb polyclectrolyte is MasterGlenium 7500.

FIG. 3: Complex coacervate emulsions stabilize upon addition of commercial comb polyelectrolytes. Stable polyelectrolyte complex coacervate emulsions with 155.3 mM charge concentration of oppositely charged linear polyelectrolytes (1:1 charge ratio) and increasing charge concentration of different anionic comb polyelectrolytes MVA2808, MVA2500 and MVA2453 (all from BASF). Photographs taken 48 hours after mixing. Turbidity in the samples is a key factor for indicating the stability of the emulsions. The linear polyelectrolytes are polydiallydimethylammonium chloride (pDADMAC) (molecular weight 8500 g/mol) and polyacrylic acid (PAA) (molecular weight 5100 g/mol). It is proposed that commercial polycarboxylate ethers with anionic acrylic acid groups on the backbone and polyethylene oxide sidechains, corresponding to the three comb polyelectrolytes shown here as well as MasterGlenium 7500, stabilize coacervate emulsions by adsorbing on the droplet surface and inducing steric repulsion between them.

FIG. 4: Emulsification can be stimulated by dilution of concentrated single-phase solution of linear polyelectrolytes and comb polyelectrolytes. (a) Turbidity map for polyelectrolyte complex coacervates prepared at varying total linear polyelectrolyte concentrations and anionic comb polyelectrolyte concentrations. Black symbols represent clear formulations. Red symbols represent turbid solutions while green and yellow symbols represent turbidity below a cutoff of 50% and 25%, respectively. Sample labelled with a blue star at ˜1865.6 mM (˜24 wt %) linear polyelectrolyte concentration with ˜84.3 mM comb polyelectrolyte concentration is chosen as the system for dilution experiments. (b) Dilution of single-phase solution comprising oppositely charged linear polyelectrolyte and anionic comb polyelectrolyte into stable complex coacervate emulsions following 12-, 24-, 30-48-, and 120-fold dilutions. Photographs are taken 48 hours after dilution. Total linear polyelectrolyte concentration and comb polyelectrolyte concentration of the diluted samples are indicated. (c) A comparison of diluted unstabilized (1′-3′) and stabilized (1-3) complex coacervate formulations, beginning with single-phase solution comprising oppositely charged linear polyelectrolyte (1865.6 mM charge concentration) and a single-phase solution comprising a mixture of oppositely charged linear polyelectrolyte (1865.6 mM charge concentration) and anionic comb polyelectrolyte (84.3 mM charge concentration). Photographs taken 48 hours after dilution. Turbidity in the samples is a key factor for indicating the stability of the emulsions. The linear polyelectrolytes are polydiallydimethylammonium chloride (pDADMAC) (molecular weight 8500 g/mol) and polyacrylic acid (PAA) (molecular weight 5100 g/mol). The comb polyelectrolyte is MasterGlenium 7500.

FIG. 5: Comb polyelectrolyte stabilized complex coacervate emulsions can withstand addition of salt. (a) Stable polyelectrolyte complex coacervate emulsions comprising oppositely charged linear polyelectrolytes (1:1 charge ratio, 62.2 mM charge concentration), anionic comb polyelectrolyte (4.8 mM charge concentration) and increasing concentration of salt (NaCl). Photographs taken at different times, indicated as “as mixed”, 3 hours, 24 hours, and 48 hours after mixing. Turbidity in the samples is a key factor for indicating the stability of the emulsions (b) Turbidity measurements indicating stability of the complex coacervate emulsions as a function of salt concentration. With increasing comb polyclectrolyte concentration (at constant linear polyelectrolyte concentration), larger salt concentrations are required to reduce the turbidity of the emulsions. (c) Micrographs of the as-prepared unstable complex coacervates droplets (column 1) and stabilized complex coacervate emulsions (column 2). The stabilized emulsions contain 4.8 mM comb polyelectrolytes in addition to the oppositely charged linear polyelectrolytes (62.2 mM charge concentration). Salt concentration is varied from 0 mM to 200 mM. Both as-mixed solutions contain micron-sized droplets. The unstable emulsions undergo droplet coalescence, while the stabilized coacervate emulsions maintain droplet stability, as evident from micrographs of the stabilized emulsions visualized after 48 hours after mixing (column 3). Scale bars are provided in each micrograph. The linear polyelectrolytes are polydiallydimethylammonium chloride (pDADMAC) (molecular weight 8500 g/mol) and polyacrylic acid (PAA) (molecular weight 5100 g/mol). The comb polyelectrolyte is MasterGlenium 7500.

FIG. 6: Comb polyelectrolytes significantly expand the coacervation window. Addition of comb polyelectrolytes increases both the critical linear polyelectrolyte concentration and the critical salt concentration at which coacervates transition into a single-phase solution. Turbidity maps of formulations comprising varying concentrations of oppositely charged linear polyelectrolytes (1:1 charge ratio) and salt concentration (NaCl) for three different anionic comb polyelectrolyte concentrations (C_cPE=0, 4.8 and 9.5 mM) highlight this expansion of the coacervation window. Black crosses represent for the clear phase systems. The solid red circles represent turbid solutions while yellow circles represent partially turbidity solutions. The linear polyelectrolytes are polydiallydimethylammonium chloride (pDADMAC) (molecular weight 8500 g/mol) and polyacrylic acid (PAA) (molecular weight 5100 g/mol). The comb polyelectrolyte is MasterGlenium 7500.

FIG. 7: Salt can provide another handle to tune microdroplet size in complex coacervate emulsions. (a-c) The evolution of the mean diameter (by volume) of the complex coacervate microdroplets in formulations comprising oppositely charged linear polyelectrolytes (1:1 charge ratio, 62.2 mM charge concentration), anionic comb polyelectrolyte (4.8 mM or 9.5 mM charge concentration) and salt. The mean size of the comb polyelectrolyte stabilized coacervate microdroplets increases with increasing salt concentration but remain nearly constant with time up to 48 hours. The microdroplet diameters were measured using dynamic light scattering and averaged from triplicate samples at each concentration and three measurement trials for each sample. Filled symbols denote data measured from unperturbed samples. Open symbols denote data measured from samples that were shaken gently. Errors are calculated through the mean standard deviations of the measurements. Error bars are shown when they are larger than the symbols. The linear polyelectrolytes are polydiallydimethylammonium chloride (pDADMAC) (molecular weight 8500 g/mol) and polyacrylic acid (PAA) (molecular weight 5100 g/mol). The comb polyelectrolyte is MasterGlenium 7500.

FIG. 8: Comb polyelectrolyte stabilized complex coacervate emulsions efficiently encapsulate proteins from solution (a) Encapsulation of proteins in stabilized complex coacervate microdroplets comprising oppositely charged linear polyelectrolytes (1:1 charge ratio, 15.9-62.2 mM charge concentration) and anionic comb polyelectrolyte (4.8 mM charge concentration) with molar ratio of protein:total polyclectrolyte=0.15. Protein loaded stable complex coacervate emulsions are visualized “as mixed” (columns 1-3) and 48 hours after mixing (columns 4-6). The comb polyelectrolytes used to stabilize the complex coacervate emulsions do not impede the protein encapsulation ability of the complex coacervates. (b) The evolution of the mean diameter (by volume) of protein containing complex coacervate microdroplets in formulations comprising oppositely charged linear polyelectrolytes (1:1 charge ratio) and anionic comb polyelectrolyte. The mean size of protein-containing microdroplets were similar to those without any protein, and the droplets remain stable up to 48 hours. The microdroplet diameters were measured using dynamic light scattering and averaged from triplicate samples at each concentration and three measurement trials for each sample. Filled symbols denote data measured from unperturbed samples. Open symbols denote data measured from samples that were shaken gently. Errors are calculated through the mean standard deviations of the measurements. Error bars are shown when they are larger than the symbols. The linear polyelectrolytes are polydiallydimethylammonium chloride (pDADMAC) (molecular weight 8500 g/mol) and polyacrylic acid (PAA) (molecular weight 5100 g/mol). The comb polyelectrolyte is MasterGlenium 7500. The protein is FITC-labelled Bovine Serum Albumin (BSA).

FIG. 9: Salt does not impede the encapsulation of proteins in stabilized complex coacervate emulsions. Encapsulation of protein in unstable and stabilized complex coacervate microdroplets comprising oppositely charged linear polyelectrolytes (1:1 charge ratio, 62.2 mM charge concentration) and stabilized by anionic comb polyelectrolyte (4.8 mM charge concentration) with molar ratio of protein:total polyclectrolyte=0.15 as a function of salt concentration. Protein loaded complex coacervate emulsions are visualized “as mixed” (columns 1-3 and 4-6) and 48 hours after mixing (columns 7-9). The comb polyelectrolytes used to stabilize the coacervate emulsions do not inhibit protein encapsulation. Moreover, comb polyelectrolyte stabilized coacervates form and can encapsulate proteins at higher salt concentrations compared to the unstable complex coacervates. The linear polyelectrolytes are polydiallydimethylammonium chloride (pDADMAC) (molecular weight 8500 g/mol) and polyacrylic acid (PAA) (molecular weight 5100 g/mol). The comb polyelectrolyte is MasterGlenium 7500. The protein is FITC-labelled Bovine Serum Albumin (BSA).

FIG. 10: Activity of enzymes is markedly enhanced upon encapsulation in stable complex coacervate microdroplets. (a) The enzymatic activity of lipase is detected by the absorbance spectra of the p-nitrophenol produced upon enzymatic degradation of p-nitrophenyl butyrate. Lipase enzymes are either suspended in solution, encapsulated in unstable complex coacervates (with total PE concentration=15.9 mM), or encapsulated in stabilized complex coacervate microdroplets composed of oppositely charged linear polyelectrolytes (with total PE concentration=15.9 mM) and anionic comb polyelectrolytes. The absorbance spectra evolved at a significantly faster rate, denoting enhanced enzyme activity in solutions where enzymes were encapsulated in stabilized microdroplets. (b) The enzymatic activity (1/t_1/2), estimated from the absorbance spectra, for freely suspended and encapsulated enzymes quantifies the activity enhancements upon enzyme encapsulation. The linear polyelectrolytes are polydiallydimethylammonium chloride (pDADMAC) (molecular weight 8500 g/mol) and polyacrylic acid (PAA) (molecular weight 5100 g/mol). The comb polyclectrolyte is MasterGlenium 7500.

FIG. 11: Highlights of our strategy to create stable complex coacervate emulsions. (a) Complex coacervates obtained upon mixing of aqueous solutions of oppositely charged linear polyelectrolytes, leading to turbidity in the as-mixed solutions emerging from the presence of droplets (column 1). These complex coacervate microdroplets are prone to coalescence and merge into a homogenous transparent coacervate phase after ˜3 hours (column 2). Stable complex coacervate emulsions are turbid when prepared (column 3) and maintain uniform turbidity for more than 48 hours (column 4). Over 4 months of storage, the complex coacervate microdroplets settle to the bottom of the vial but do not coalesce, leading to a dense emulsion at the bottom of the vial (column 5). Upon gentle shaking of the vial, the microdroplets re-disperse, resulting in turbid emulsions (column 6). The shaken vials remain turbid for the next 48 hours (column 7). (b) Micrographs of the as-prepared unstable complex coacervates droplets (column 1) and stabilized complex coacervate emulsions as-prepared (column 2) and after 48 hours after mixing (column 3). (c) Dilution of a single-phase solution comprising oppositely charged linear polyelectrolyte and anionic comb polyelectrolyte into stable complex coacervate emulsions following 12-, 24-, 30-48-, and 120-fold dilutions. Photographs were taken 48 hours after dilution. (d) Encapsulation of proteins in stabilized complex coacervate microdroplets comprising oppositely charged linear polyelectrolytes and comb polyelectrolyte with FITC-BSA. (e) The enzymatic activity for freely suspended and encapsulated lipase quantifies the activity enhancements upon encapsulation in stable complex coacervate microdroplets. For (a-e), the linear polyelectrolytes are polydiallydimethylammonium chloride (pDADMAC) (molecular weight 8500 g/mol) and polyacrylic acid (PAA) (molecular weight 5100 g/mol). The comb polyelectrolyte is MasterGlenium 7500. The protein in (d) is FITC-labelled Bovine Serum Albumin (BSA). The enzyme in (e) is lipase.

FIG. 12: Quantification of the evolution of emulsion turbidity and droplet size demonstrates exceptional stability. (a, b) Turbidity evolution, demonstrated as a function of time after mixing, of the stabilized complex coacervate emulsions (filled symbols) and the unstable complex coacervates (open symbols) are illustrated for formulations comprising oppositely charged linear polyelectrolytes (1:1 charge ratio) and stabilized by anionic comb polyelectrolyte (4.8 mM charge concentration). The linear polyclectrolyte charge concentrations are (a) 38.8 mM and (b) 72.2 mM. The turbidity of stabilized complex coacervate emulsions remained >95% for over 48 hours while that of unstable complex coacervates reduced by 90%. (c) Evolution of mean diameter of the microdroplets in the stabilized complex coacervates, demonstrated as a function of time after mixing, for formulations comprising oppositely charged linear polyelectrolytes (1:1 charge ratio) and stabilized by anionic comb polyclectrolyte (4.8 mM charge concentration). The linear polyelectrolyte charge concentrations are 38.8 mM (red symbols) and 72.2 mM (blue symbols). For (a)-(c), the linear polyelectrolytes are polydiallydimethylammonium chloride (pDADMAC) (molecular weight 8500 g/mol) and polyacrylic acid (PAA) (molecular weight 5100 g/mol). The comb polyclectrolyte is MasterGlenium 7500.

FIG. 13: Droplet size distribution in stabilized complex coacervate emulsions. (a-d) Distribution of the microdroplet diameter, by volume, are shown for stabilized complex coacervate emulsions in as-mixed samples and samples incubated for 48 hours after mixing. The formulations comprised oppositely charged linear polyelectrolytes (1:1 charge ratio) and stabilized by anionic comb polyelectrolyte (4.8 mM charge concentration). The linear polyclectrolyte charge concentrations are 38.8 mM (a,b) and 72.2 mM (c,d). Narrow size distributions of the microdroplets are demonstrated in all samples. The microdroplet size increased with increasing linear polyclectrolyte concentration. The microdroplets size increased marginally after 48 hours while still remaining below 2 μm. Different colors represent results from repeated measurements. The linear polyelectrolytes are polydiallydimethylammonium chloride (pDADMAC) (molecular weight 8500 g/mol) and polyacrylic acid (PAA) (molecular weight 5100 g/mol). The comb polyclectrolyte is MasterGlenium 7500.

FIG. 14: Turbidity maps of complex coacervate formulations comprising comb polyelectrolyte. The green shading highlights the coacervation window in each figure. (a) Turbidity map for polyelectrolyte complex coacervates prepared at varying total linear polyelectrolyte concentrations and anionic comb polyelectrolyte concentrations. Black symbols represent clear formulations. Red symbols represent turbid solutions while green and yellow symbols represent turbidity below a cutoff of 50% and 25%, respectively. (b-d) Turbidity maps of formulations comprising varying concentrations of oppositely charged linear polyelectrolytes (1:1 charge ratio) and salt concentrations (NaCl) for three different anionic comb polyelectrolyte concentrations (C_cPE=0 (b), 4.8 (c) and 9.5 mM (d)) highlight this expansion of the coacervation window. The addition of comb polyelectrolytes increases both the critical linear polyelectrolyte concentration and the critical salt concentration at which coacervates transition into a single-phase solution. Black crosses represent the clear phase systems. The solid red circles represent turbid solutions while yellow circles represent partially turbidity solutions. The linear polyelectrolytes are polydiallydimethylammonium chloride (pDADMAC) (molecular weight 8500 g/mol) and polyacrylic acid (PAA) (molecular weight 5100 g/mol). The comb polyelectrolyte is MasterGlenium 7500.

FIG. 15: Strong polyelectrolyte pair coacervate microdroplets formed at high concentrations of KBr can be stabilized by comb polyelectrolytes. Electrostatic interactions between strongly and oppositely charged polyelectrolytes polydiallyldimethylammonium chloride (molecular weight 8500 g/mol) and polystyrene sulfonate (molecular weight 70,000 g/mol) were screened by introducing salt KBr (concentration of 1.8M); this plasticized the complexes formed to yield liquid-like coacervates instead of solid-like precipitates. Emulsions of coacervates were prepared (1:1 charge ratio, 136 mM linear polyelectrolyte charge ratio) and stabilized by the addition of increasing concentration of anionic comb polyelectrolyte from 11.7-58.8 mM charge concentration. Photographs of these taken at different times, indicated as “as mixed”, “24 hrs” and “48 hrs” after mixing are displayed. Turbidity in samples indicates the presence of coacervate microdroplets, and its persistence at different times shows that the emulsions are stable. KBr is known to screen electrostatic interactions more effectively than NaCl, and emulsions maintain turbidity after 48 hrs at high comb-polyelectrolyte concentrations (58.8 mM) despite high KBr concentration (1.8M). The comb polyelectrolyte used was polyacrylic acid-co-polyethylene glycol (PAA-co-PEG, molecular weight=39467 g/mol, 26 e− at pH=6, PEG molecular weight=3000 g/mol, commercial name MasterGlenium 7500)

FIG. 16: Encapsulation of enzymes in stabilized complex coacervates can enable biocatalysis in flow reactors. Comb polyelectrolyte stabilized coacervate droplet emulsion with encapsulated protein lipase was placed in a continuous stir tank reactor (CSTR) to demonstrate its utility in flow reactors. Dialysis membranes were used to restrict these micron sized droplets within the reactor volume while allowing the free flow of the reactants and products. As shown earlier, encapsulated lipase catalyzes the degradation of p-nitrophenyl butyrate (PNPB) to the fluorescent p-nitrophenol at increased rates. PNPB was added to the CSTR inlet feed on day 1, day 5 and day 21, and the outlet concentration of p-nitrophenol was obtained from the absorbance at its characteristic wavelength. As expected with the unsteady CSTR operation during start-up, the conversion at reactor outlet shows an initial positive slope followed by a decay on day 1. Conversion of day 5 and day 21 plateau to similar values, showing that the enzymatic activity lipase is maintained, thereby demonstrating the feasibility of using such emulsions in flow reactors. For this experiment, CSTR volume=0.55 L, flow rate=0.28 ml/s, residence time ˜35 min and lipase concentration=0.05 μM. The coacervate phase was composed of polydiallyldimethylammonium chloride (molecular weight 8500 g/mol) and polyacrylic acid (molecular weight 5100 g/mol) with total charge concentration of 40 mM, and comb polyclectrolyte polyacrylic acid-co-polyethylene glycol (PAA-co-PEG, molecular weight=39467 g/mol, 26 e⁻ at pH=6, PEG molecular weight=3000 g/mol, commercial name MasterGlenium 7500) with concentration of 23.5 mM charge.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the aspects of the techniques and procedures described or referenced herein are well understood and commonly employed by those skilled in the art. The following text discusses various embodiments of the invention.

A variety of materials with diverse structures and properties can be formed as a result of electrostatic interactions between oppositely charged macromolecules. Under defined conditions, complexation between oppositely charged polyelectrolytes can lead to a phase separation phenomenon, referred to as complex coacervation. Polyelectrolyte complex (PEC) coacervates form upon electrostatic complexation of oppositely charged macromolecules and their subsequent condensation into aqueous macromolecule-rich phases. These aqueous two-phase systems have been demonstrated to possess unique capabilities to achieve dynamic spatial compartmentalization and spontaneous sequestering of biological molecules (see, e.g., Thies, in Encyclopedia of Food Sciences and Nutrition (Second Edition), 2003; Shadell et la., Food Hydrocolloids Volume 77, April 2018, Pages 803-816; U.S. Pat. No. 9,757,333; and U.S. Patent Application Publication No. 20090253165). Despite such exciting prospects, their use has been limited owing to an inability to stabilize coacervate droplets and prevent their macro-phase separation.

Complex coacervates can form upon electrostatic complexation of oppositely charged macromolecules (synthetic polyelectrolytes, biopolyelectrolytes like DNA and RNA, charged biomacromolecules such as proteins, macroions, etc.). Their associative liquid-liquid phase separation spontaneously results into two phases, with one macromolecule-rich phase containing both polyelectrolytes and the other being primarily water. These coacervate droplets possess distinct water-water interfaces with the ambient aqueous environments and have a strong propensity to partition and encapsulate charge-bearing molecules (e.g., proteins, multivalent ions, nucleic acids and the like). As such, these aqueous two-phase systems have been presented as minimalistic membraneless protocellular model that exhibit unique capabilities to achieve dynamic spatial compartmentalization as well as spontaneous sequestering and activity enhancement of biological molecules. Despite these exciting prospects, their utility as protocells, bioreactors, and encapsulants have been significantly hampered owing to the lack of colloidal stability, which makes the coacervate droplets prone to coalescence and Ostwald ripening, leading to macrophase separation of complex coacervates.

Studies of water-water emulsions have achieved stabilization of aqueous droplets by encapsulation with membranes composed of hydrophilic-hydrophobic-charged triblock polyelectrolytes that prevent droplet condensation into macrophases. Similar stabilization strategies have also been accomplished for complex coacervate droplets by self-assembly of lipid vesicles on the droplet surfaces wherein the vesicles tend to adsorb at the water-water interfaces to provide colloidal stability. However, these approaches create a membrane around the droplets that is semi-permeable at best. Therefore, key attributes of complex coacervate-based bioreactors including strong propensity to sequester charged macromolecules from solution and active transport of small molecules in and out of the droplets are severely diminished.

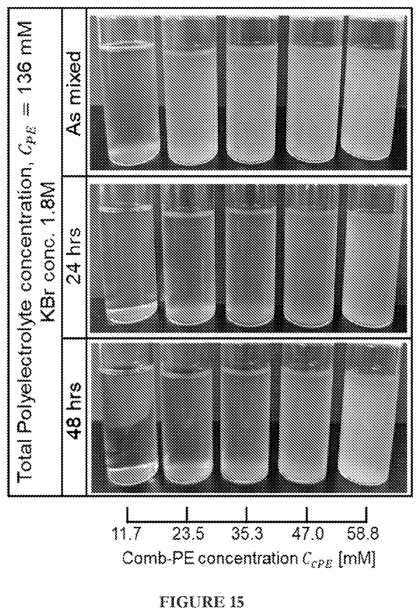

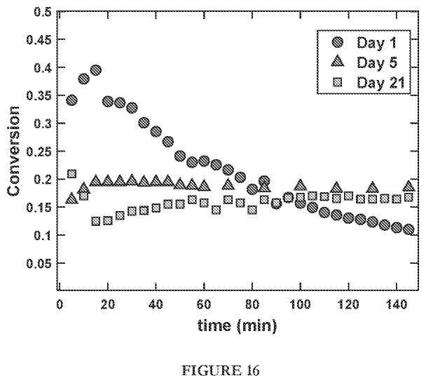

Comb polymers are a class of branched polymers consisting of a linear backbone with a low grafting density of side chains. Polyelectrolytes are a group of polymers whose repeating units bear an electrolyte group. These groups dissociate in aqueous solutions (water), making the polymers charged. As disclosed herein, complex coacervates composed of a charged polyelectrolyte pair of molecules can be stabilized with comb polyelectrolytes at high salt concentrations and electrostatic screening. At low ionic strengths, strongly charged polyelectrolyte pairs typically form solid-like precipitates, and high salt concentrations are required to screen coulombic interactions to yield liquid-like coacervates. In illustrative embodiments, we use potassium bromide for effective electrostatic screening. Such coacervate droplets can also be stabilized by addition of comb-polyelectrolytes and remain dispersed after 48 hours. This shows that stabilization of coacervates using comb-polyelectrolytes is a versatile strategy which is effective for high ionic strengths, distinct salt identities, and different polyelectrolyte strengths (see, e.g., FIG. 15). Stabilized coacervate emulsions with encapsulated enzymes can also be used effectively for biocatalysis in continuous flow reactors at practical scales. A ˜0.5 L reactor with ˜0.3 ml/min flow rate loaded with such emulsions to perform enzyme catalyzed reactions is shown to maintain its activity after 21 days of its start-up. Conversion at the outlet is also observed to remain constant at steady state operation over this period (See, e.g., FIG. 16).

The invention described herein provides methods and materials that can impart long-term stability to polyelectrolyte complex coacervate droplets and create complex coacervate emulsions. Embodiments of the invention include, for example, compositions of matter including water; a water-soluble comb polyclectrolyte; a positively charged water-soluble macromolecule; and a negatively charged water-soluble macromolecule; wherein the water-soluble comb polyelectrolyte, the positively charged water-soluble macromolecule and the negatively charged water-soluble macromolecule form a complex coacervate. In typical embodiments of the invention, the water-soluble comb polyelectrolyte comprises an anionic comb polyelectrolyte; the positively charged water-soluble macromolecule comprises a linear polyelectrolyte; and the negatively charged water-soluble macromolecule comprises a linear polyelectrolyte. In certain embodiments of the invention, the composition further comprises at least one additional agent comprising: a pesticide; a polypeptide; a polynucleotide; a therapeutic agent; a diagnostic agent; a macroion; a pharmaceutical excipient; and/or a salt. Optionally such compositions are formed so that the additional agent exhibits a charge concentration such that the ratio of the additional agent's charge to the sum of the positively charged macromolecule charge concentration and the negatively charged macromolecule charged concentration ranges from 0 to 0.3.

In some embodiments of the invention, the complex coacervate comprises microdroplets having a mean diameter from 0.05 μm to 10 μm. In some embodiments of the invention, the complex coacervate exhibits turbidity at concentrations of 100, 250 or 500 mM NaCl. In some embodiments of the invention, the complex coacervate remains stable for 12, 24 or 48 hours following a >10-fold, >50-fold or >120-fold dilution from a concentrated single-phase solution of the complex coacervate. In some embodiments of the invention, the complex coacervate spontaneously encapsulates polypeptides disposed in the composition. In some embodiments of the invention, the composition comprises microdroplets; and the microdroplets remain suspended in the composition for 12, 24 or 48 hours following microdroplet formation; and/or the mean diameter of the microdroplets remains stable for 24 or 48 hours following microdroplet formation; and/or the mean diameter of the microdroplets remains stable for 24 or 48 hours in concentrations of 100 mM NaCl. In some embodiments of the invention, the positively charged water-soluble macromolecule and the negatively charged water-soluble macromolecule exhibit a charge ratio of 0.25:1-4:1 (e.g. 1:1). In some embodiments of the invention, the positively charged water-soluble macromolecule is at a concentration from 0.1-50 wt %. In some embodiments of the invention, the negatively charged water-soluble macromolecule is at a concentration from 0.1-50 wt %. In some embodiments of the invention, the comb polyelectrolyte exhibits a charge concentration such that the ratio of the comb polyclectrolyte charge concentration to the sum of the positively charged macromolecule charge concentration and the negatively charged macromolecule charged concentration ranges from 0.05 to 1. In some embodiments of the invention, the positively charged water-soluble linear polyelectrolyte is present in amounts from 0.1 mM-3000 mM. In some embodiments of the invention, the negatively charged water-soluble linear polyelectrolyte is present in amounts from 0.1 mM-3000 mM. In some embodiments of the invention, the water-soluble comb polyelectrolyte is present in amounts of at least 0.1 mM. In some embodiments of the invention, sodium chloride is present in amounts from 1 to 1000 mM.

A variety of polycations, polyanions and comb polyelectrolytes can be used in embodiments of the invention (see, e.g. Table 1). For example, in addition to the polycation polydiallyldimethylammonium chloride, other polycations such as polyallylamine hydrochloride, poly(ethyleneimine), poly(vinylamine), cationic polyacrylamides and naturally sourced polycations such as chitosan and cationic starches can be used to form coacervate droplets. In addition to polyanions such as polyacrylic acid and polystyrene sulfonate, other groups of polyanions such as polycarboxylates, polyphosphonates, polysulphonates, anionic polyacrylamides and naturally sourced polyanions such as sulphated polysaccharides and ligin sulphonates can be used to form coacervate droplets. In addition to comb polyelectrolytes such as polyacrylic acid-co-polyethylene glycol which has a polyacrylic acid backbone, all the above mentioned charged polycations and polyanions may constitute the polymer backbone. Instead of polyethylene glycol, the neutral side chains can be composed of any other hydrophilic polymer such as polyacrylamides as well as natural neutral polymers such as starch and cellulose derivatives.

The complex coacervate formulations are typically composed of oppositely charged linear polyelectrolytes (that form the complex coacervate phase) and water. Stability against coalescence of droplets is achieved by incorporation of comb polyelectrolytes in the formulations, leading to complex coacervate emulsions. Salt is added optionally to tune the properties of the emulsions. Proteins are added optionally, for example to create bio-microreactors. In embodiments of the invention, the comb polyelectrolytes adsorb on the complex coacervate droplet's surfaces (water-water interfaces) and provide steric stabilization to the droplets. In this context, the microdroplet size and number density can be adjusted by varying the total polyelectrolyte concentration, comb polyelectrolyte concentration, and optionally salt concentration. Different embodiments of the invention use different sized molecules depending on the use/purpose for the composition. In certain embodiments of the invention, the backbone of the water-soluble comb polyelectrolyte and/or the backbone of the positively charged water-soluble macromolecule (e.g linear polyelectrolyte) and/or the backbone of the negatively charged water-soluble macromolecule (e.g linear polyelectrolyte); comprises at least 10², 10³, 10⁴, 10⁵, 10⁶, or 10⁷ monomeric units covalently linked together to form the backbone. In other embodiments of the invention, the backbone of the water-soluble comb polyelectrolyte and/or the backbone of the positively charged water-soluble macromolecule (e.g linear polyelectrolyte) and/or the backbone of the negatively charged water-soluble macromolecule (e.g linear polyelectrolyte); comprises less than 10², 10³, 10⁴, 10⁵, 10⁶, or 10⁷ monomeric units covalently linked together to form the backbone.

The complex coacervate emulsion compositions of the invention can include additional components such as at least one of: a polypeptide; a polynucleotide; a therapeutic agent; a diagnostic agent; a macroion; a pharmaceutical excipient; and/or sodium chloride (i.e., microdroplet cargos, and excipients, and the like). Certain embodiments of the methods and compositions of the invention include, for example the use of a pharmaceutical excipient such as one selected from the group consisting of a preservative (e.g., an antimicrobial agent), a polypeptide stabilizing agent, a tonicity adjusting agent, a detergent, a viscosity adjusting agent, a sugar and a pH adjusting agent. For compositions suitable for administration to humans, the term “excipient” is meant to include, but is not limited to, those ingredients described in Remington: The Science and Practice of Pharmacy, Lippincott Williams & Wilkins, 21st ed. (2006) the contents of which are incorporated by reference herein.

A variety of compositions can be made using the methods and materials disclosed herein. In embodiments of the invention, the compositions can have varying amounts of ingredients, for example ones where the positively charged water-soluble linear polyelectrolyte is present in amounts from 0.1 mM-3000 mM; and/or the negatively charged water-soluble linear polyelectrolyte is present in amounts from 0.1 mM-3000 mM; and/or the water-soluble comb polyelectrolyte comprising a negatively charged backbone and neutral sidechains is present in an amount of at least 0.1 mM (e.g. in amounts from 1 mM-500 mM). In illustrative embodiments of the invention, in the complex coacervate emulsion compositions, the positively charged water-soluble linear polyclectrolyte is present in amounts from 0.4 mM-1150 mM; the negatively charged water-soluble linear polyclectrolyte is present in amounts from 0.4 mM-1150 mM; the water-soluble comb polyelectrolyte comprising a negatively charged backbone and neutral sidechains is present in amounts from 4.8 mM-84.6 mM; and/or sodium chloride is present in amounts from 1 to 1000 mM.

The compositions disclosed herein can formed to exhibit a number of structural and/or functional properties. For example, in certain embodiments of the invention, the composition comprises microdroplets having a mean diameter from 0.05 μm to 10 μm (e.g. from 0.2 μm to 2 μm); and/or the complex coacervate emulsion composition exhibits turbidity at concentrations of 100, 250 or 500 mM NaCl; and/or the complex coacervate emulsion remains stable for 12, 24 or 48 hours following a >120-fold dilution from a concentrated single-phase solution of the complex coacervate emulsion; and/or the complex coacervate emulsion spontaneously encapsulates polypeptides disposed in the composition. In certain embodiments of the invention, the microdroplets remain suspended in solution for 12, 24 or 48 hours following microdroplet formation; and/or the mean diameter of the microdroplets remains stable for 24 or 48 hours (or 1 week or 1 month) following microdroplet formation; and/or the mean diameter of the microdroplets remains stable for 24 or 48 hours (or 1 week or 1 month) in concentrations of 100 mM NaCl.

Embodiments of the invention also include methods of making complex coacervates having the constellation of elements disclosed herein. Typically, these methods comprise combining together water; a water-soluble comb polyelectrolyte (e.g., an anionic comb polyelectrolyte); a positively charged water-soluble macromolecule (e.g. a first linear polyelectrolyte); and a negatively charged water-soluble macromolecule (e.g. a second linear polyelectrolyte). In such methods, the water-soluble comb polyelectrolyte, the positively charged water-soluble macromolecule and the negatively charged water-soluble macromolecule are selected to have material properties and used in concentrations/ratios that allow them to be combined together in the water to form a complex coacervate. Some embodiments of these methods include further disposing in the complex coacervate at least one of: a polypeptide; a polynucleotide; a therapeutic agent; a diagnostic agent; a macroion; a pesticide; a pharmaceutical excipient; a salt or the like. In illustrative methods of making a complex coacervate, the comb polyelectrolyte is selected to adsorb on complex coacervate droplet surfaces (water-water interfaces) so as to provide steric stabilization to the droplets. In certain of the methods of making a complex coacervate the method forms microdroplets having a mean diameter from 0.05 μm to 10 μm; and/or the complex coacervate formed in the method exhibits turbidity at concentrations of 100, 250 or 500 mM NaCl; and/or the complex coacervate formed in the method remains stable for 12, 24 or 48 hours following a >120-fold dilution from a concentrated single-phase solution of the complex coacervate. In some embodiments of the invention, the complex coacervate formed in the method spontaneously encapsulates polypeptides disposed in the composition. Embodiments of the invention further include complex coacervates made by the methods disclosed herein.

The methods of the invention can be used to form compositions having selected properties. For example, in some embodiments of the invention, microdroplets formed by the method remain suspended in solution for 12, 24 or 48 hours following microdroplet formation. In some embodiments of the invention, the mean diameter of the microdroplets formed by the method remains stable for 24 or 48 hours following microdroplet formation. In some embodiments of the invention, the mean diameter of the microdroplets formed by the method remains stable for 24 or 48 hours in concentrations of 100, 200 or 300 mM NaCl. In certain embodiments of the invention, the positively charged water-soluble macromolecule and the negatively charged water-soluble macromolecule are selected to exhibit a charge ratio of 0.25:1-4:1 (e.g. 1:1). In some embodiments of the invention, the positively charged water-soluble macromolecule is at a concentration from 0.1-50 wt %; the negatively charged water-soluble macromolecule is at a concentration from 0.1-50 wt %; and the comb polyelectrolyte is selected to exhibit a charge concentration such that the ratio of the comb polyclectrolyte charge concentration to the sum of the positively charged macromolecule charge concentration and the negatively charged macromolecule charged concentration ranges from 0.05 to 1.

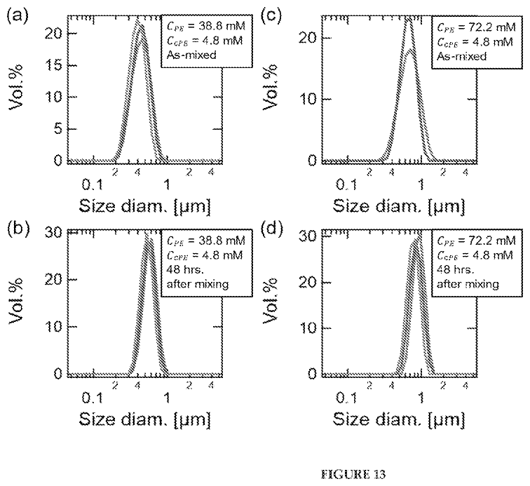

Embodiments of the invention include methods of making a complex coacervate emulsion comprising combining together: a positively charged water-soluble linear polyelectrolyte; a negatively charged water-soluble linear polyelectrolyte; an anionic water-soluble comb polyelectrolyte (e.g., one comprising a negatively charged backbone and neutral sidechains); and water, such that a complex coacervate emulsion is made. In certain embodiments of the invention, these methods include disposing in the reaction mixture combination at least one of: a polypeptide; a polynucleotide; a protein; a therapeutic agent; a diagnostic agent; a macroion; a pharmaceutical excipient; and/or salts such as sodium chloride. Typically, in such embodiments, the method forms microdroplets having a mean diameter from 0.2 μm to 2 μm. The mean droplet size and the size distribution varies as a function of linear polyelectrolyte (C_PE) and comb polyclectrolyte (C_cPE) concentrations. FIG. 13 shows the droplet size distribution for a few representative stabilized complex coacervate emulsions, with C_PE=38.8 mM (a,b) or C_PE=72.2 mM (c,d) and C_cPE=4.8 mM. This width of the droplet size distribution can be tuned by adjusting C_PE and C_cPE.

In certain embodiments of the invention, the complex coacervate emulsion composition formed in the method exhibits turbidity at concentrations of 100, 250 or 500 mM NaCl; the complex coacervate emulsion formed in the method remains stable for 12, 24 or 48 hours following a >120-fold dilution from a concentrated single-phase solution of the complex coacervate emulsion; and/or the complex coacervate emulsion formed in the method spontaneously encapsulates polypeptides disposed in the composition. In certain embodiments of the invention, microdroplets formed by the method remain suspended in solution for 12, 24 or 48 hours following microdroplet formation; and/or the mean diameter of the microdroplets formed by the method remains stable for 24 or 48 hours following microdroplet formation; and/or the mean diameter of the microdroplets formed by the method remains stable for 24 or 48 hours in concentrations of 100, 200 or 300 mM NaCl.

In certain embodiments of the invention, in the complex coacervate emulsion made by these methods, the positively charged water-soluble linear polyelectrolyte is present in amounts from 0.1 mM-3000 mM (e.g. from 0.4 mM-1150 mM); the negatively charged water-soluble linear polyelectrolyte is present in amounts from 0.1 mM-3000 mM (e.g. from 0.4 mM-1150 mM); the water-soluble comb polyelectrolyte comprising a negatively charged backbone and neutral sidechains is present in an amount of at least 0.1 mM (e.g. from 4.8 mM-84.6 mM); and/or a salt such as sodium chloride is present in amounts from 1 to 1000 mM.

Embodiments of the invention also include methods of using the compositions disclosed herein. For example, embodiments of the invention include methods of using the compositions disclosed herein to stabilize an activity of a molecule and/or inhibit the degradation of the molecule. For example, the near-native coacervate environment of the compositions disclosed herein can stabilize encapsulated protein against denaturation due to temperature changes. Proteins in solution are usually stored in refrigerated conditions, which increases their storage cost. Encapsulation of proteins in coacervate droplets can allow their storage at room temperatures and reduce costs.

A typical embodiment of the invention is a method of stabilizing an activity of a biological molecule such as a polypeptide (e.g., an enzyme such as lipase) and/or inhibiting the degradation of this molecule, the method comprising disposing the biological molecule such as a polypeptide in a microdroplet of the invention such that an activity of this cargo is stabilized and/or its degradation is inhibited (i.e. as compared to a control cargo within the same environment but not disposed within a microdroplet of the invention). Such methods comprise disposing the molecule as a cargo in a microdroplet of a complex coacervate composition disclosed herein, such that an activity of the cargo molecule is stabilized and/or its degradation is inhibited.

Embodiments of the invention also include methods of performing a biochemical reaction, the methods comprising disposing molecules that perform the biological reaction (e.g. one or more enzymes such as an oxidoreductase; a transferase; a hydrolase; a lipase; a isomerase or a ligase) as a cargo within a complex coacervate formed to comprise water; a water-soluble comb polyelectrolyte; a positively charged water-soluble macromolecule; and a negatively charged water-soluble macromolecule; such that the biochemical reaction is performed within the complex coacervate.

The complex coacervate emulsions-based bioreactors disclosed herein can also support multi-enzyme reaction cascades by co-encapsulating more than 1 enzyme in the droplets. Multiple enzymes may also be encapsulated in an individual coacervate droplet, thereby allowing enzymatic cascades wherein the product from one enzyme is the reactant for another. In this context because the enzymes are in proximity inside the droplet, mass transfer limitations will be reduced significantly, and overall reaction rates will be much higher. Such embodiments of the invention allow for the construction of artificial enzymatic cascades, providing great flexibility. In certain embodiments of the invention, the cargo comprises a plurality of enzymes; and the product generated by a first enzyme in the plurality of enzymes is a reactant for a second enzyme within the plurality of enzymes (e.g., so as to perform an enzyme cascade within the complex coacervate composition).

Embodiments of the invention include methods of delivering a cargo (e.g., a polypeptide; a polynucleotide; a protein; a therapeutic agent; a diagnostic agent or the like) to a patient, the method comprising administering the cargo disposed in a microdroplet within a composition disclosed herein to the patent such the cargo is delivered to an in vivo location in the patient. In alternative embodiments of the invention, the composition is adapted for an agricultural application and includes a cargo stabilized by the complex coacervates disclosed herein (e.g. an agrochemical such as a pesticide, or an herbicide or a fertilizer or the like) adapted for this purpose (see WO 2017029302 and U.S. Patent Publications 20181039956 and 20110294864, and Liu et al., Advanced Functional Materials (31):5 2021, Hynes et al., Weed Technology Vol. 24, No. 2 (APRIL-JUNE 2010), pp. 185-192, and Huang et al., Nanomaterials 2018, 8(2), 102 which are incorporated by reference). In alternative embodiments of the invention, the composition is part of a detergent composition and includes a cargo (e.g., an enzyme such as a protease, an amylase, a lipase, a cellulase, or a mannanase) adapted for this purpose.

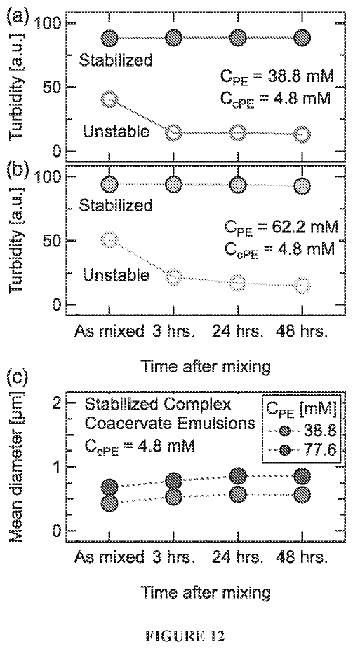

As noted above, in certain embodiments of the invention, the complex coacervate emulsion is “stable” (e.g., stable for 12, 24 or 48 hours following a >120-fold dilution). In such embodiments of the invention, “stable” can be defined as situations where the mean diameter of the microdroplets in the composition does not change or changes minimally. Typically, for example. “stable” typically denotes a situation where the mean size diameter of the microdroplets in the composition changes less than 20%, less than 10% or less than 5%. As shown in FIG. 12, the mean diameter of stabilized complex coacervate emulsion droplets (FIG. 12C) remains constant for at least 48 hours after mixing while the unstable droplets phase separates into a macroscopic single phase. This data shows that the mean droplet size does not change in the stabilized emulsion, indicating lack of coalescence of droplets.

As noted above, in certain embodiments of the invention, the complex coacervate emulsion exhibits “turbidity”. Turbidity of unstable coacervates and stabilized complex coacervate emulsions is shown in FIGS. 12A and 12B. The stabilized complex coacervate emulsions maintain turbidity at almost 100% up to 48 hours, while the unstable complex coacervates become clear, with more than 80% loss in turbidity in the same timeframe. Turbidity here is defined as (1−fraction of incident light transmitted through the sample)*100%. So, a 100% turbid sample does not let any light through while a 0% turbid sample is completely transparent. In certain embodiments of the invention where a complex coacervate emulsion “maintains turbidity” under a particular condition (such as varying salt concentrations), this means that this emulation maintains at least 50% of the original turbidity observed in a control complex coacervate emulsion not exposed to that condition (e.g., a particular salt concentration).

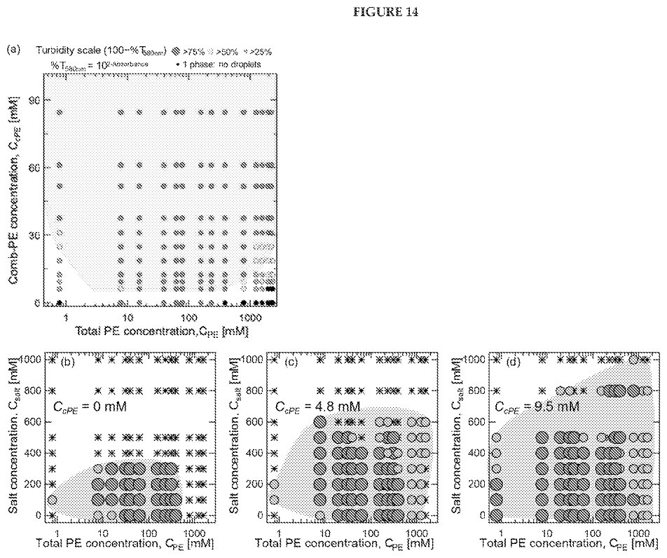

Illustrative concentration boundaries that we have observed form stable complex coacervate emulsions that maintain turbidity up to 48 hours after mixing are as follows (and data showing the stability window for samples without salt is depicted in FIG. 14A and data showing the stability window for samples with salt is depicted in FIGS. 14B-D):

Samples without salt:
Linear polyelectrolyte concentration: 0.8 mM-2300 mM
Positively charged linear polyelectrolyte concentration: 0.4 mM-1150 mM
Negatively charged linear polyelectrolyte concentration: 0.4 mM-1150 mM
Comb polyelectrolyte concentration: 0.9 mM-84.6 mM

Samples with salt:
Linear polyelectrolyte concentration: 0.8 mM-2000 mM
Positively charged linear polyelectrolyte concentration: 0.4 mM-1000 mM
Negatively charged linear polyelectrolyte concentration: 0.4 mM-1000 mM
Comb polyelectrolyte concentration: 0, 4.8 mM, 9.5 mM
Sodium chloride concentration: 0 to 1000 mM
   In FIG. 14, the linear and comb polyelectrolyte concentrations are described in terms of their net charge concentrations. The droplets with proteins are also stable in similar concentration ranges as those without proteins, as discussed above.

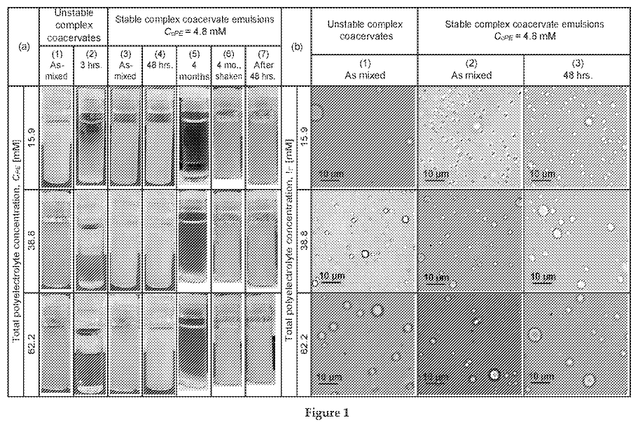

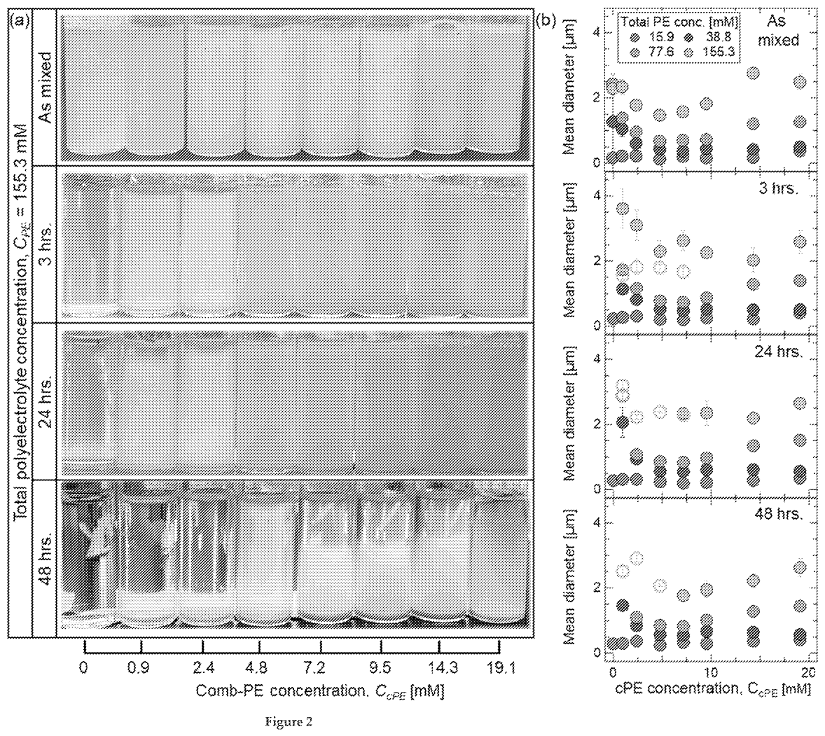

In embodiments of the invention, long-term stability of the complex coacervate emulsions is established by pronounced turbidity of the emulsions after 4+ months. While unstable complex coacervate microdroplets coalesce within 3 hours, stabilized droplets remain suspended in solution up to 48 hours. At 4 months, the droplets may settle to the bottom but redisperse upon gentle shaking of the vials, resulting in turbid solutions (FIGS. 1 and 2). The complex coacervate macrophase that forms upon coalescence of unstable complex coacervate microdroplets does not redisperse into the microdroplets upon shaking. In embodiments of the invention, control over microdroplet size is attained by varying the total linear polyelectrolyte concentration and comb polyelectrolyte concentrations. The mean size of the microdroplets remains stable up to 48 hours after preparation of the emulsions (FIG. 2).

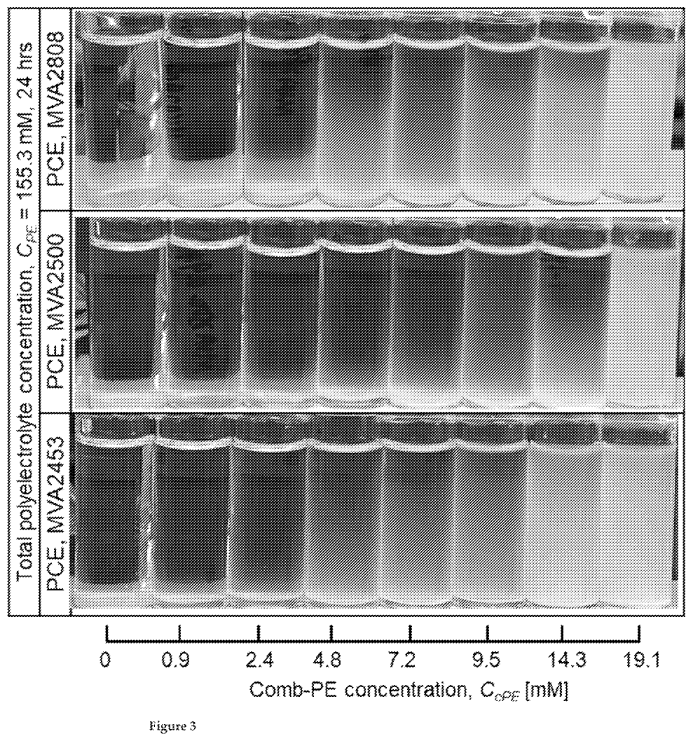

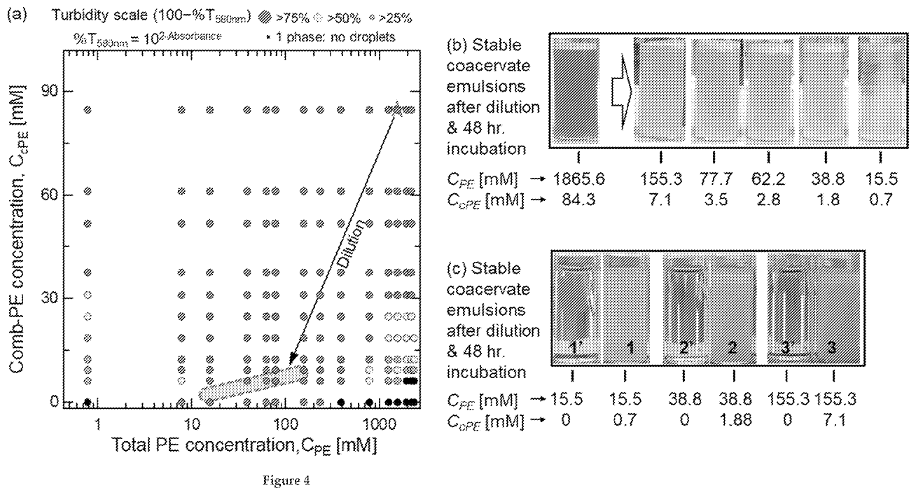

In embodiments of the invention, stable complex coacervate emulsion formulations can be achieved with commercially available linear and comb polyelectrolytes. 4 different comb polyelectrolytes are demonstrated to provide stable emulsions, with varying degrees of stability. (FIG. 3). The complex coacervate emulsion formulations are stable against dilution. Complex coacervate emulsions remain stable for up to 48 hours after >120-fold dilution from a concentrated single-phase solution. The corresponding unstable complex coacervate solutions coalesce readily upon dilution from a macrophase separated coacervate (FIG. 4).

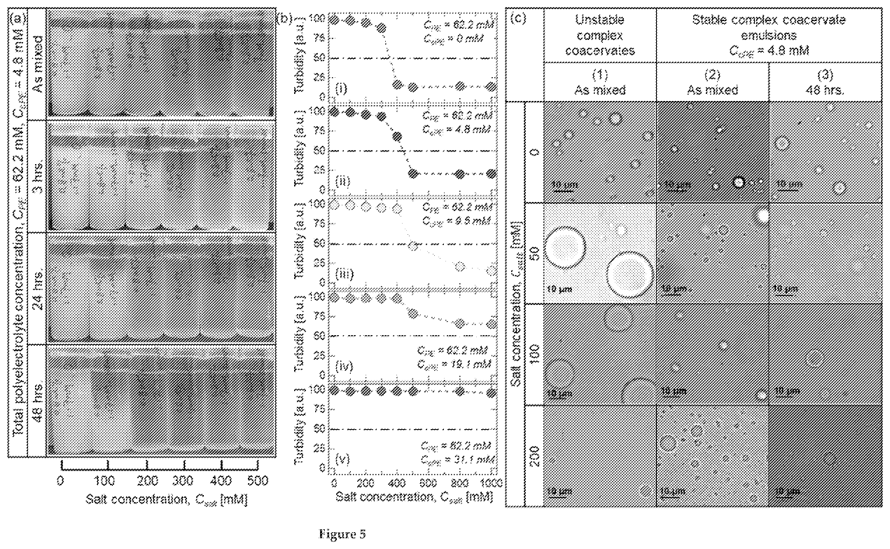

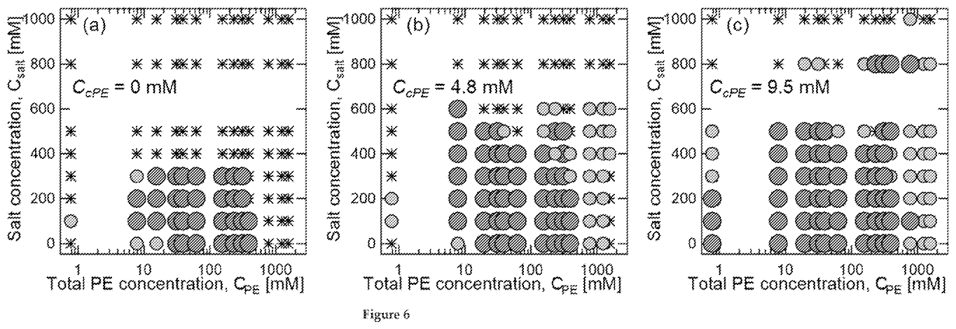

The stabilization strategy involving comb polyelectrolytes is resistant to the addition of salt in the complex coacervate solutions. Typically, the addition of salt results in dissolution of the complex coacervate phase above a critical salt concentration. However, comb polyelectrolyte-stabilized complex coacervate emulsion formulations exhibit turbidity even up to 500 mM NaCl concentrations. The critical salt concentration and the critical total linear polyelectrolyte concentration at which the complex coacervate phase vanishes both increases upon incorporation of comb polyelectrolytes in the formulations. (FIGS. 5 and 6).

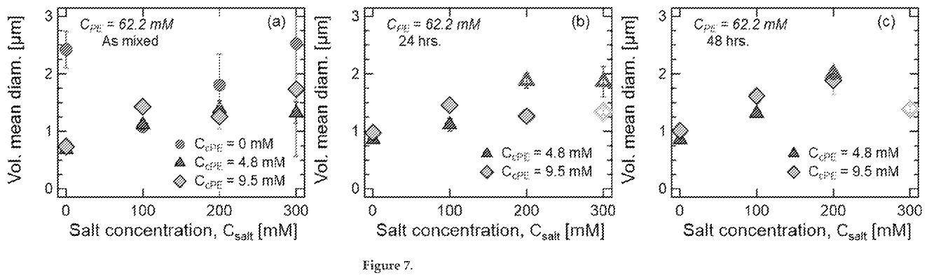

The size of comb polyelectrolyte-stabilized complex coacervate microdroplets increases with an increasing salt concentration in solution. The stabilized microdroplets remain stable up to 48 hours against coalescence in presence of 300 mM salt, signifying that the stabilizing mechanism is robust and can withstand the presence of high concentrations of salt. (FIG. 7)

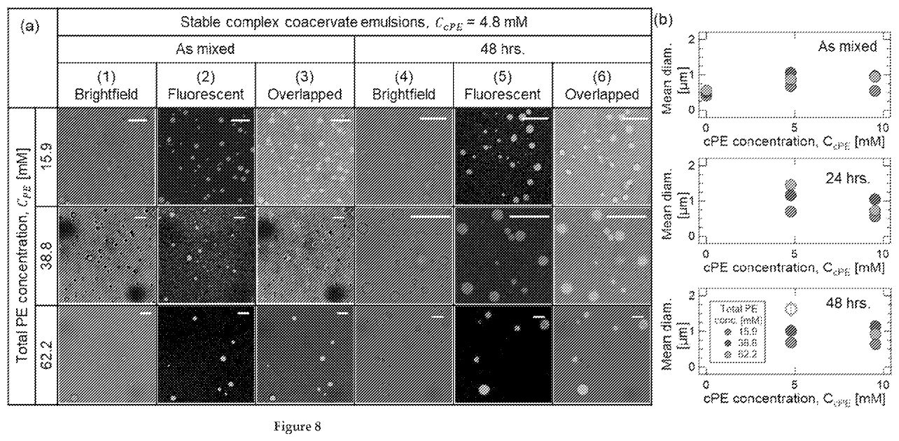

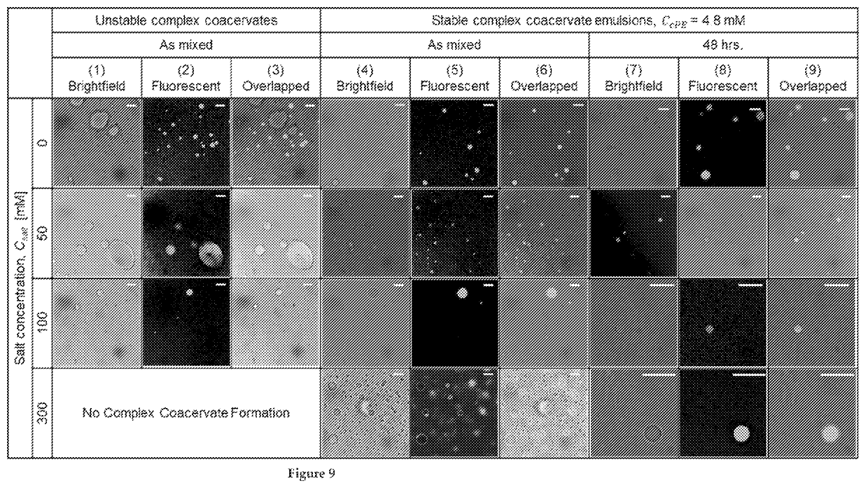

The complex coacervate emulsion formulations spontaneously encapsulate proteins introduced in the solution. A strong partitioning of proteins (fluorescently labeled Bovine Serum Albumin [BSA]) into the microdroplets can be achieved by varying the total linear polyelectrolyte concentration and comb polyelectrolyte concentrations. The size of the protein containing microdroplets remains stable up to 48 hours. (FIG. 8). Salt concentrations up to 300 mM do not impede the partitioning of proteins (fluorescently labeled Bovine Serum Albumin [BSA]) into the complex coacervate microdroplets. (FIG. 9)

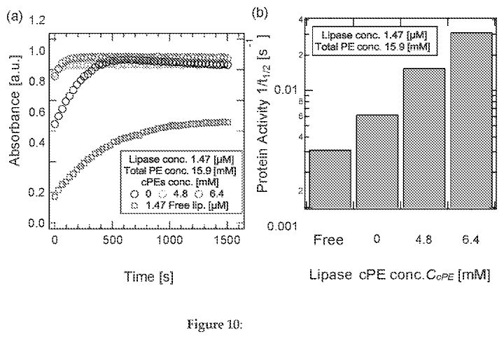

Encapsulation of enzymes in the stabilized complex coacervate microdroplets enhances their enzymatic activity significantly. A 10-fold enhancement in the activity of an enzyme (lipase) encapsulated in stable complex coacervate microdroplets is achieved in comparison to the activity of free enzymes in solution. Moreover, this enhancement in enzyme activity represents a >5-fold improvement in comparison to the activity of enzymes encapsulated in unstable complex coacervate droplets. (FIG. 10)

Embodiments of the invention disclosed herein include coacervate emulsions stabilized by comb-polyelectrolytes (cPE), such as those that are commercially used as dispersants. We demonstrate that the stable complex coacervate emulsions exhibit long-term (>4-month) stability, composed of oppositely charged linear polyelectrolytes and stabilized by the polycarboxylate ether-based comb-polyelectrolyte interfacially adsorbing at the coacervate-water interfaces. The microdroplet size remains relatively constant with time and is shown to be regulated by the concentrations of the comb- and linear polyelectrolytes. The emulsion properties can be optionally tuned by the addition of water and salt while the stability is not influenced. This stabilization strategy provides steric stabilization to the droplets while allowing the transport of small molecules in and out of the coacervate droplets and retaining the tendency to sequester proteins and other charged molecules from solution, paving the way for them to be immensely employed as multifunctional bioreactors and encapsulants in agricultural, cosmetics, food, and pharmaceutical formulations.

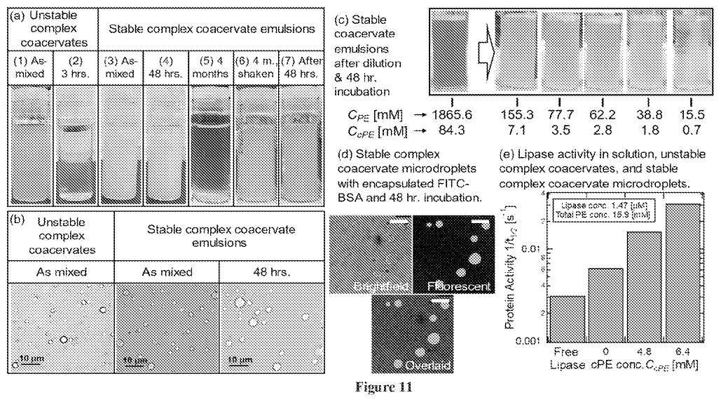

As disclosed herein, we have developed methods for making stable complex coacervate emulsions that imparts long-term stability to complex coacervate droplets while conserving their membraneless attributes. Stabilization of complex coacervate microdroplets composed of oppositely charged linear polyelectrolytes can be demonstrated upon introduction of comb-polyelectrolytes (cPE) promoted by their interfacial adsorption at the coacervate-water interfaces. Stabilized microdroplets that remain suspended in solution for up to 48 hours can be contrasted against unstable complex coacervate microdroplets that coalesce within 3 hours after mixing of the oppositely charged polyelectrolytes (FIGS. 11a and 11b). Over 4 months, the stabilized microdroplets may settle to the bottom of the vials but redisperse upon gentle shaking, resulting in turbid solutions (FIG. 11a). In contrast, the complex coacervate macrophase that forms upon coalescence of unstable complex coacervate microdroplets does not redisperse even upon vigorous shaking. Moreover, the microdroplet size remains relatively stable with time and is shown to be regulated by the concentration ratio of the comb- and the linear polyelectrolytes. Our stabilization strategy is amenable to excessive dilution of the emulsions (FIG. 11c) and robust against addition of salt, making it particularly appealing to consumer care product formulations. At the same time, tunability of the stability of the microdroplets can be modulated by varying attributes of the comb-polyelectrolytes.

The stabilization methodology we adopt does not interfere with the sequestration of proteins into the complex coacervate droplets, paving way for creation of self-assembled microdroplets that provide protective and supportive environments for proteins, enzymes, and other charge-bearing macromolecules. Encapsulation of proteins in the stabilized complex coacervate microdroplets is demonstrated with a representative system of fluorescently-labeled bovine serum albumin (FIG. 11d). Protein encapsulation efficiency of the coacervate droplets will be shown, in both salt-free and salt solutions, to be minimally influenced by the presence of comb polyclectrolyte stabilizers. Moreover, prominent improvements in enzymatic activity upon encapsulation of the enzymes in the stabilized complex coacervate droplets, as compared to their activity when suspended in solution or encapsulated in unstable complex coacervate droplets, will be demonstrated. Successful acceleration of lipase-mediated hydrolysis of p-nitrophenyl butyrate to p-nitrophenol in stabilized microdroplets will illustrate both the activity enhancements of the enzyme as well as facile transport of small molecules substrates and products in and out of the complex coacervate microdroplets (FIG. 11c). In summary, we have demonstrated a framework for creation of complex coacervate based encapsulants, stabilizers, microreactors and the like.

### RELEVANT PUBLICATIONS

1. Panova, Irina G., Dolgor D. Khaydapova, Leonid O. Ilyasov, Aminat B. Umarova, and Alexander A. Yaroslavov. “Polyclectrolyte complexes based on natural macromolecules for chemical sand/soil stabilization.” Colloids and Surfaces A: Physicochemical and Engineering Aspects 590 (2020): 124504.
2. Panova, I. G., L. O. Ilyasov, D. D. Khaidapova, A. S. Bashina, A. V. Smagin, K. Ogawa, Y. Adachi, and A. A. Yaroslavov. “Soil conditioners based on anionic polymer and anionic micro-sized hydrogel: A comparative study.” Colloids and Surfaces A: Physicochemical and Engineering Aspects 610 (2021): 125635.
3. Stewart, Russell John. “Reinforced adhesive complex coacervates and methods of making and using thereof” U.S. patent application Ser. No. 13/114,397, filed Nov. 24, 2011.
4. Scheuing, David R., David J. Lestage, Carl W. Bennett, Mona M. Knock, Charles W. Scales, William L. Smith, and Rui Zhang. “Polyelectrolyte Complexes.” U.S. patent application Ser. No. 12/749,288, filed Sep. 29, 2011.
5. Nguyen-Kim, S., Muller, G., Wood, C. and Hossel. P., 2006. Cosmetic and pharmaceutical substances based on polyelectrolyte complexes. U.S. patent application Ser. No. 10/564,627.

Additional materials and methods that can be adapted for use with embodiments of the invention include those disclosed in US Patent Publication Nos. 20210084953; 20200215232; 20200054786; 20180360976; 20180071185; 20170043197; 20160008475; 20150104545; 20130004617; 20120201748; 20110294864; 20110083681; 20080075778; 20080044480; 20070010408; 20060293213; 20060293197; 20060276371; 20060275337; 20060134282; and 20020006886, the contents of which are incorporated by reference.

All publications mentioned herein (e.g., those listed above) are incorporated herein by reference to disclose and describe aspects, methods and/or materials in connection with the cited publications.

TABLE 1
Illustrative comb polyelectrolytes useful in embodiments of the invention.

| | | Side chain molecular | Charge | Charge | |
|---|---|---|---|---|---|
| | Molecular | weight (g/mol) | density at | density at | Rapid |
| | weight | (side chain monomer | pH = 6 | pH = 12 | solids |
| Brand name | (g/mol) | units) | (e⁻/mol) | (e⁻/mol) | content |
| MasterGlenium7500 | 394 | 3000 | 25.7 | 43.4 | 26.59% |
| | 67 | (68) | | | |
| BASF | 265 | 1100 | 31.5 | 47.3 | 44.03% |
| MVA2453 | 28 | (25) | | | |
| BASF | 490 | 5800 | 25.2 | 52.4 | 44.43% |
| MVA2500 | 75 | (132) | | | |
| BASF | 219 | 1100/3000 | 32.8 | 47.0 | 51.20% |
| MVA2808 | 66 | (25/68) | | | |

1. A composition of matter comprising:
water;
a water-soluble comb polyelectrolyte;
a positively charged water-soluble macromolecule; and
a negatively charged water-soluble macromolecule;
wherein the water-soluble comb polyelectrolyte, the positively charged water-soluble macromolecule and the negatively charged water-soluble macromolecule form complex coacervate droplets.

2. The composition of claim 1, wherein:
the water-soluble comb polyelectrolyte comprises an anionic comb polyelectrolyte;
the positively charged water-soluble macromolecule comprises a linear polyelectrolyte; and
the negatively charged water-soluble macromolecule comprises a linear polyelectrolyte.

3. The composition of claim 2, wherein:
the positively charged water-soluble macromolecule and the negatively charged water-soluble macromolecule exhibit a charge ratio of 0.25:1-4:1;
the positively charged water-soluble macromolecule is at a concentration from 0.1-50 wt %;
the negatively charged water-soluble macromolecule is at a concentration from 0.1-50 wt %;
the comb polyelectrolyte exhibits a charge concentration such that the ratio of the comb polyelectrolyte charge concentration to the sum of the positively charged macromolecule charge concentration and the negatively charged macromolecule charged concentration ranges from 0.05 to 1; and/or
(e) the composition further comprises at least one additional agent comprising: a pesticide; a polypeptide; a polynucleotide; a protein; a therapeutic agent; a diagnostic agent; a macroion; a pharmaceutical excipient; and/or a salt.

4. The composition of claim 3, wherein the additional agent exhibits a charge concentration such that the ratio of the additional agent's charge to the sum of the positively charged macromolecule charge concentration and the negatively charged macromolecule charged concentration ranges from 0 to 0.3.

5. The composition of claim 2, wherein:
the complex coacervate comprises microdroplets having a mean diameter from 0.05 μm to 10 μm;
the complex coacervate exhibits turbidity at concentrations of 100, 250 or 500 mM NaCl;
the complex coacervate remains stable for 12, 24 or 48 hours following a >120-fold dilution from a concentrated single-phase solution of the complex coacervate; and/or
the complex coacervate spontaneously encapsulates polypeptides disposed in the composition.

6. The composition of claim 2, wherein composition comprises microdroplets; and
the microdroplets remain suspended in the composition for 12, 24 or 48 hours following microdroplet formation;
the mean diameter of the microdroplets remains stable for 24 or 48 hours following microdroplet formation; and/or
the mean diameter of the microdroplets remains stable for 24 or 48 hours in concentrations of 100 mM NaCl.

7. The composition of claim 2, wherein:
the positively charged water-soluble linear polyelectrolyte is present in amounts from 0.1 mM-3000 mM;
the negatively charged water-soluble linear polyelectrolyte is present in amounts from 0.1 mM-3000 mM;
the water-soluble comb polyelectrolyte is present in amounts of at least 0.1 mM; and/or
sodium chloride is present in amounts from 1 to 1000 mM.

8. A method of making a complex coacervate comprising combining together:
water;
a water-soluble comb polyelectrolyte;
a positively charged water-soluble macromolecule; and
a negatively charged water-soluble macromolecule;
wherein the water-soluble comb polyelectrolyte, the positively charged water-soluble macromolecule and the negatively charged water-soluble macromolecule are selected for their ability to combine together in the water to form a complex coacervate;
such that a complex coacervate is made.

9. The method of claim 8, wherein:
the water-soluble comb polyelectrolyte comprises an anionic comb polyelectrolyte;
the positively charged water-soluble macromolecule comprises a linear polyelectrolyte; and
the negatively charged water-soluble macromolecule comprises a linear polyelectrolyte.

10. The method of claim 8, further comprising disposing in the complex coacervate at least one of:
a pesticide;
a polypeptide;
a polynucleotide;
a therapeutic agent;
a diagnostic agent;
a macroion;
a pharmaceutical excipient; and/or
a salt.

11. The method of claim 8, wherein:
the comb polyelectrolyte is selected to adsorb on complex coacervate droplet surfaces so as to provide steric stabilization to the droplets;
the method forms microdroplets having a mean diameter from 0.05 μm to 10 μm;
the complex coacervate formed in the method exhibits turbidity at concentrations of 100, 250 or 500 mM NaCl;
the complex coacervate formed in the method remains stable for 12, 24 or 48 hours following a >120-fold dilution from a concentrated single-phase solution of the complex coacervate; and/or
the complex coacervate formed in the method spontaneously encapsulates polypeptides disposed in the composition.

12. The method of claim 11, wherein:
microdroplets formed by the method remain suspended in solution for 12, 24 or 48 hours following microdroplet formation;
the mean diameter of the microdroplets formed by the method remains stable for 24 or 48 hours following microdroplet formation; and/or
the mean diameter of the microdroplets formed by the method remains stable for 24 or 48 hours in concentrations of 100, 200 or 300 mM NaCl.

13. The method of claim 8, wherein:
the positively charged water-soluble macromolecule and the negatively charged water-soluble macromolecule are selected to exhibit a charge ratio of 0.25:1-4:1;
the positively charged water-soluble macromolecule is at a concentration from 0.1-50 wt %;
the negatively charged water-soluble macromolecule is at a concentration from 0.1-50 wt %;
the comb polyelectrolyte is selected to exhibit a charge concentration such that the ratio of the comb polyelectrolyte charge concentration to the sum of the positively charged macromolecule charge concentration and the negatively charged macromolecule charged concentration ranges from 0.05 to 1.

14. A complex coacervate made by the method of claim 8.

15. A method of stabilizing an activity of a molecule and/or inhibiting the degradation of the molecule, the method comprising disposing the molecule as a cargo in a microdroplet of the composition of claim 5, such that an activity of the cargo molecule is stabilized and/or its degradation is inhibited.

16. The method of claim 15, wherein the cargo molecule performs a chemical reaction within the composition.

17. A method of performing a biochemical reaction, the method comprising disposing molecules that perform the biological reaction as a cargo within a complex coacervate comprising:
water;
a water-soluble comb polyelectrolyte;
a positively charged water-soluble macromolecule; and
a negatively charged water-soluble macromolecule;
such that the biochemical reaction is performed within the complex coacervate.

18. The method of claim 17, wherein the cargo comprises an enzyme.

19. The method of claim 18, wherein the enzyme is an oxidoreductase; a transferase; a hydrolase; a lyase; an isomerase or a ligase.

20. The method of claim 18, wherein:
the cargo comprises a plurality of enzymes; and
the product generated by a first enzyme in the plurality of enzymes is a reactant for a second enzyme within the plurality of enzymes.
